(19) United States
(12) Patent Application Publication
(10) Pub. No.: US 2012/0184944 A1
(43) Pub. Date: Jul. 19, 2012

(54) Coiled Tubing for Drain Bag Applications

(76) Inventors: Jennifer E. Tomes, Mundelein, IL (US); Sarah C. Zyburt, Chicago, IL (US); James E. Burgess, Mundelein, IL (US); John Henry Kutsch, Harvard, IL (US)

(21) Appl. No.: 13/009,613

(22) Filed: Jan. 19, 2011

(51) Int. Cl. A61M 27/00

(52) U.S. Cl. 604/544

(57) ABSTRACT

A catheter assembly (200) includes drain tubing (201) extending from a catheter connector (203) to a drain bag (202). The drain tubing (201) includes a first elongated section (205), a helical section (206), and a second elongated section (207). The helical section (206) can be configured to be expanding and self-retracting, thereby eliminating dependent loops that lead to pooled fluids or air-fluid locks. A hanger (215) can be configured with a mechanical connection to help align and stiffen the drain tubing (201). A valve (212) can be color-coded to indicate whether it is open or closed.

### BACKGROUND

1. Technical Field

This invention relates generally to catheter systems, and more particularly to a tubing apparatus and drain bag suitable for use as a Foley catheter assembly.

2. Background Art

Conventional catheter devices employ a thin sterile tube that is inserted into a patient for the purpose of draining bodily fluids. For example, a Foley catheter includes a thin tube that is inserted into a patient's bladder for the purpose of draining urine. The urine flows through the tube and generally collects in a drain bag attached to the tube opposite the patient.

Turning to FIG. 1, illustrated therein is one example of a prior art Foley catheter system 100. These systems can be used when a patient 101 is confined to a bed 102 or is otherwise unable to go to the restroom. The catheter tube 103 is inserted through the patient's urethra into the bladder. The catheter tube 103 is then held in place by a balloon that is filled with sterile water. The catheter tube 103 drains urine into a drain bag 104 that is coupled to the patient's bed 102. A health care provider can then empty the contents of the drain bag 104 into the proper receptacle when full.

A frequent problem with prior art catheter systems 100 involves the catheter tube 103. As shown in the expanded view 105 in FIG. 1, the catheter tube frequently forms a “dependent loop” 106, in which a portion 107 of the catheter tube 103 falls below the fluid entry point 108 of the drain bag 104. As shown in FIG. 1, the dependent loop 106 resembles a “U-shape,” although it may take other geometric shapes as well. When this occurs, urine 109 and other fluids can pool in the dependent loop 106. This is problematic because pooled urine 109 can be a source of microbial growth, which can lead to infection. Further, an air-fluid lock can develop which places backpressure against subsequently flowing urine. This backpressure can result in the patient's bladder being forced to store newly produced urine. Where either occurs, the patient 101 becomes at risk for urinary tract infections.

It would be beneficial to have a catheter assembly capable of overcoming these issues.

### BRIEF DESCRIPTION OF THE DRAWINGS

FIG. 1 illustrates a prior art catheter system and the problematic dependent loop that can form.

FIG. 2 illustrates a catheter system configured in accordance with one or more embodiments of the invention.

FIG. 3 illustrates one embodiment of a hanger for a drain bag suitable for use with one or more embodiments of the invention.

FIG. 4 illustrates a perspective view of a catheter system configured in accordance with one or more embodiments of the invention.

FIGS. 5, 6, and 7 illustrate a top plan, front section elevation, and side section elevation views, respectively, of a catheter system configured in accordance with one or more embodiments.

FIG. 8 illustrates a front elevation view of one embodiment of a drain bag configured in accordance with one or more embodiments of the invention.

FIGS. 9 and 10 illustrate embodiments of color-coded tap valves and instructions corresponding thereto in accordance with one or more embodiments of the invention.

FIG. 11. illustrates one embodiment of a catheter assembly being used to prevent the formation of dependent loops in accordance with one or more embodiments of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF EMBODIMENTS OF THE INVENTION

Embodiments of the invention are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions. Also, reference designators shown herein in parenthesis indicate components shown in a figure other than the one in discussion. For example, talking about a device (10) while discussing figure A would refer to an element, 10, shown in figure other than figure A.

As noted above, when a dependent loop of tubing forms in drain bag tubing, problems can arise. This is due in part to the fact that fluid passing through the tubing is forced to flow against gravity to reach the distal end of the tube. The formation of dependent loops is frequently due to the fact that the tubing must be sufficiently long to permit patient movement. When the drain bag is coupled to a bed, walker, rolling stand, or other device, and is not fully using the length of the tubing, excess tubing is falls into one or more dependent loops. As patients come in different sizes and shapes, and as patients reside in differently shaped devices (i.e., beds, wheelchairs, etc.), it is not possible to custom fit the tubing length to prevent the formation of dependent loops. Prior art attempts to do so have resulted in limited patient mobility and movement, discomfort, and other problems.

Embodiments of the invention provide a robust solution to the dependent loop problem by including a helical section in the tubing material of a catheter system. In one embodiment, the helical section is configured as a spring coil that is integrated with substantially straight sections of tubing. In one embodiment the helical section can expand and contract, thereby letting the overall tubing length change without the formation of dependent loops. This provides the patient with adequate mobility without the risk of pooled urine or air-fluid locks. In one embodiment, the helical section is operable with a bag hanger configured to stiffen and retain the helical section in an upright position such that fluids passing through the tubing always flow in the direction of gravity. Accordingly, a drain bag connected to the tubing with the helical section can be placed in different locations without losing the advantage of gravity for the collection of fluids.

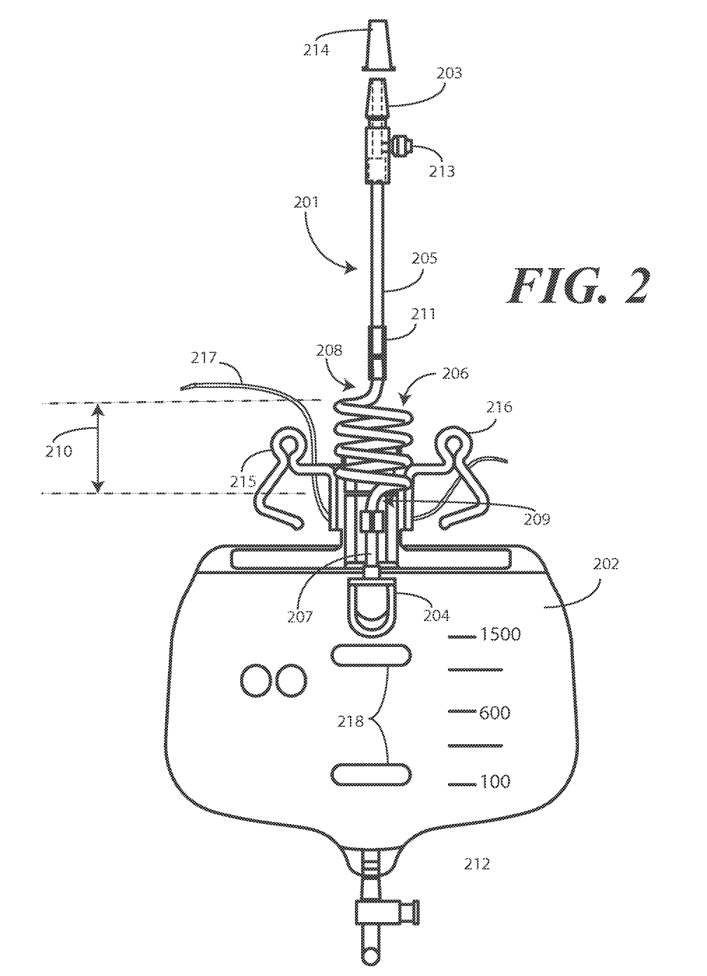

Turning now to FIG. 2, illustrated therein is one example of a catheter assembly 200 configured in accordance with one or more embodiments of the invention. The catheter assembly's primary components are drain tubing 201 and a drain bag 202. The drain tubing 201 extends from a catheter connector 203 and works as a fluid conduit connection between a catheterization device coupled to the catheter connector 203 and the drain bag 202. The drain bag 202, in one embodiment, includes an inlet connection 204 configured for attachment to the drain tubing 201.

In one embodiment, the drain tubing 201 is manufactured from a flexible tubing material. One suitable flexible tubing material is polyvinyl chloride, which is also known as “PVC.” In one embodiment, the drain tubing 201 is manufactured from 90A PVC. Other materials may used as flexible tubing material as well, including polyurethane, nylon, polyester, customized elastomers, customized polymers, thermoplastic elastomers, and so forth.

In one embodiment, the drain tubing 201 includes three sections having different geometrical configurations: a first elongated section 205, a helical section 206, and a second elongated section 207. The first elongated section extends distally from a first end 208 of the helical section 206 towards the catheter connector 203. In one embodiment, the first elongated section 205 terminates at the catheter connector 203. In another embodiment, the first elongated section 205 can include an integrated catheter assembly.

The second elongated section 207 extends distally from a second end 209 of the helical section 206 towards the drain bag 202. In one embodiment, the second elongated section 207 terminates at the inlet connection 204, which functions as a drain bag connector. The second elongated section 207 can be integrally formed with the drain bag 202. Alternatively, a selectively detachable component such as the inlet connection 204 of FIG. 2 can make the drain tubing 201 selectively detachable from the drain bag 202. This latter embodiment allows the drain tubing 201 and/or drain bag 202 to be replaced as necessary without replacing the entire catheter assembly 200.

In one embodiment, the helical section 206 comprises a plurality of turns that form a coil. For example, in the illustrative embodiment of FIG. 2, the helical section 206 comprises four turns. In other embodiments, the helical section 206 comprises between three and six turns. It will be clear to those of ordinary skill in the art having the benefit of this disclosure that other numbers of turns can be used as well. The choice in the number of turns will depend in part upon the application, in part on the geometrical shape of each of the turns, in part on the material used for the tubing, and in part on other factors. Experimental testing has shown that in a polyvinyl chloride helical section where the flexible tubing material has an outer diameter of about 6.3 millimeters, between three and six turns works well in practice.

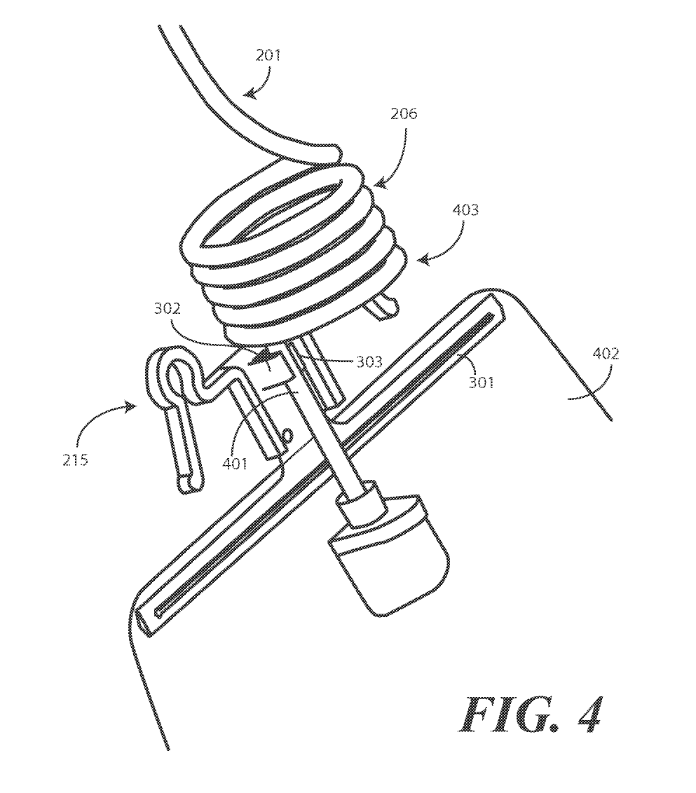

In one embodiment, the coil is configured so as to be expandable. Said differently, the length of the helical section, and thus the length of the overall connector formed by the flexible tubing material, is configured to be alterable by pulling the first elongated section 205 away from the second elongated section 207. Such a pulling action, as may be caused when a patient having the drain tubing 201 inserted in his bladder turns over, alters an axial length 210 of the helical section 206 by expanding the coil. In the illustrative embodiment of FIG. 2, the helical section 206 is formed from 90A polyvinyl chloride passing through four turns, with each turn having an outer diameter of two inches. Where so configured, the helical section 206 acts “springy” due to the material, durometer, thickness, and coil diameter. This springy nature causes the helical section 206 to be “self-retracting” in that when the pulling force is released or ceases the helical section 206 returns to a coiled state. Shown graphically, the helical section 206 of FIG. 2 is expanded, while the helical section 206 of FIG. 4 has retracted back to an initial state.

In one embodiment, the first elongated section 205, the helical section 206, and the second elongated section 207 are integrally formed together with a unitary piece of flexible tubing material. In another embodiment, one or more of the sections is formed as a separate component. Illustrating by example, in the exemplary embodiment of FIG. 2, the first elongated section 205 is a separate component from the helical section 206. In this illustrative embodiment, the first elongated section 205 is coupled to the helical section 206 by a tubular connector 211. In one embodiment, the tubular connector 211 comprises a segment of polyvinyl chloride tubing having an inside diameter on one end that substantially matches the outside diameter of the helical section 206, and an inside diameter on the other end that substantially matches the outside diameter of the first elongated section 205. As with the drain tubing 201 itself, other materials may be used for the tubular connector 211.

In one embodiment, where one or more of the first elongated section 205, the helical section 206, and the second elongated section 207 are separate, they can be configured with different inside and/or outside diameters to further enhance the flow of fluid therein. In the illustrative embodiment of FIG. 2, the first elongated section 205 is a separate component from the helical section 206. Each section has a fluid-conveying aperture therein. To function as a better connector for establishing a fluid connection between the catheter connector 203 and the drain bag 202, in one embodiment the fluid conveying apertures can have different physical dimensions that foster more efficient fluid flow.

In one embodiment, the first elongated section 205 has a fluid-conveying aperture that is greater than the fluid-conveying aperture in the helical section 206. For instance, the first elongated section 205 may have a diameter of about 6.5 millimeters while the helical section has a diameter of about 6.3 millimeters. This “wider first to narrower next” configuration works with gravity to assist the flow of fluids and prevent air-fluid locks from forming in the drain tubing 201. The different diameter of the first elongated section allows a direct flow of fluid from the patient to the helical section 206. The helical section 206 then “siphons” the fluid into the drain bag 202. The helical section 206 therefore provides a dual function of both facilitating fluid flow and enabling versatility in drain bag 202 placement. It will be clear to those of ordinary skill in the art having the benefit of this disclosure that the diameters, lengths, and dimensional differences described herein are illustrative and are not intended to be limiting.

In one embodiment, the different sections have different lengths. Experimental testing has shown that an overall drain tubing length of between about 1060 and about 1400 millimeters works well in Foley catheter applications. Accordingly, in one embodiment the first elongated section has a length of between about 1000 and 1300 millimeters. In one illustrative embodiment, the first elongated section 205 has a length of about 1170 millimeters. (The term “about” is used to include manufacturing tolerances and other variations that may occur in dimension.) In one embodiment, the helical section 206 and second elongated section 207 combine to have a length of between about 60 and 100 millimeters, and in one embodiment have a length of about 80 millimeters.

The drain bag 202 can take a variety of forms. In one embodiment, the drain bag 202 is a standard drain bag suitable for use in conventional Foley catheter applications. In another embodiment, the drain bag 202 is configured with a slightly smaller capacity than is found in traditional systems. For example, in the illustrative embodiment of FIG. 2, the drain bag 202 is configured as a lower profile bag with a capacity of about 1500 milliliters or less. This reduced capacity allows the height and/or width of the drain bag to be only about eight inches, which works to minimize the potential for the drain bag 202 to touch the ground when coupled to a patient retention device like a hospital bed.

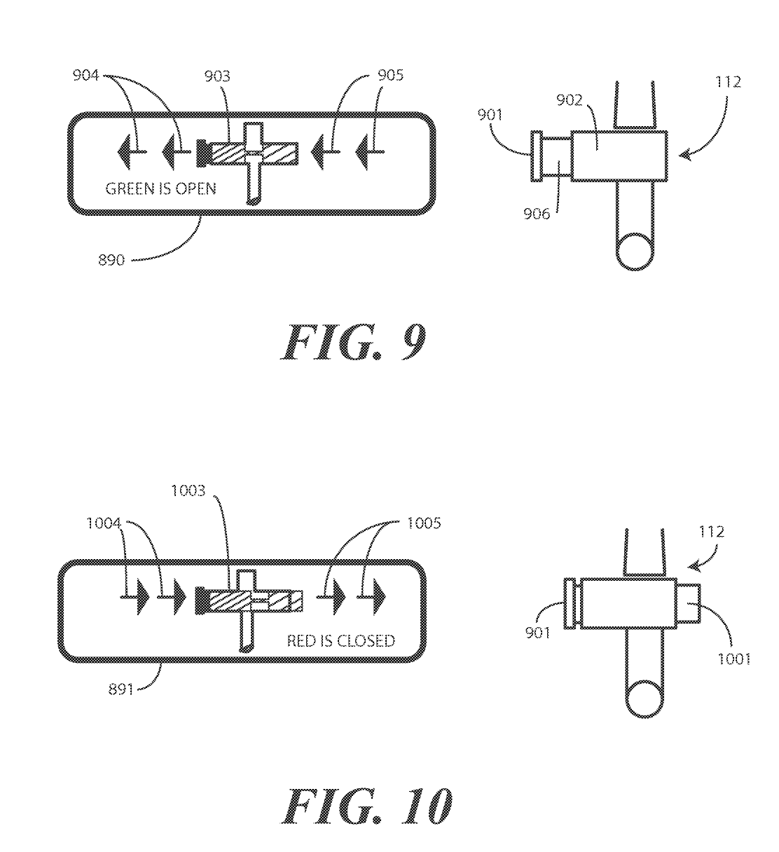

In one embodiment, the drain bag 202 includes a valve 212. The valve can be used by medical personnel for both emptying the drain bag 202 and for taking samples from the drain bag 202. In one embodiment, the valve 212 is configured as a “slide tap.” The operation of the slide tap will be explained in more detail with reference to FIGS. 9 and 10. In one embodiment, the slide tap is configured with a colored indicator that is indicative of whether the valve 212 is open. For example, in one embodiment the colored indicator can be configured to be red when the valve 212 is closed and green when the valve 212 is open. Instructions 218 regarding the color-coding system can be placed on the drain bag 202. Examples of the instructions 218 will be described below with reference to FIGS. 9 and 10. The color-coding feature makes it easier for medical personnel to know if the valve 212 is open or closed.

In one embodiment, the catheter connector 203 includes a sampling port 213. A health care services provider can take samples from the sampling port 213 by capturing fluid before it mixes with fluid in the drain bag 202. In one embodiment, the sampling port 213 is configured with a locking device, such as a Luer fitting, such that Luer-type and other syringes can be locked to the sampling port 213 when sampling is desired. In one or more embodiments, a protective cap 214 may be included with the catheter connector 203 to protect the catheter connector 203 during transit. The protective cap 214 can also help to keep the catheter connector 203 sterile prior to use. The catheter connector 203, protective cap 214, or sampling port 213 can each be manufactured from polyvinyl chloride, although other materials can also be used.

A hanger 215 can be included with the catheter assembly 200. The hanger 215 can be used, for example, to attach the drain bag 202 to a patient retention device such as a bed, chair, or wheelchair, or a patient assistance device such as a walker. The hanger 215 may be equipped with integrated clips 216 for this purpose. In some embodiments, secondary coupling devices 217 may be included as well. In the illustrative embodiment of FIG. 2, the secondary coupling devices 217 are strings suitable for tying the hanger 215 to another object.

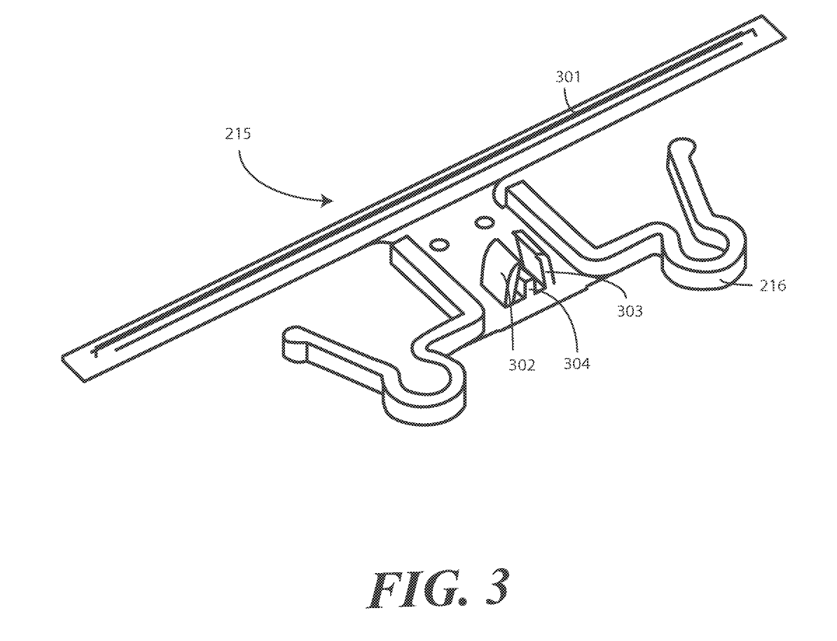

Turning to FIG. 3, illustrated therein is a perspective view of the hanger 215 of FIG. 2. The integrated clips 216 can be seen, as can the bag connection device 301. In one embodiment, the hanger 215 is manufactured from polypropylene, although other materials can be used.

In one embodiment, the hanger 215 is equipped with mechanical features that help to keep the coiled section (206) of the drain tubing (201) constantly aligned with respect to the coil. In many cases, this constant alignment will be substantially vertical when the hanger 215 is coupled to a drain bag (202). For example, when the bag connection device 301 is coupled to a drain bag, and the integrated clips 216 are attached to another object, the weight of the drain bag (202) will cause the bag connection device 301 to hang beneath the integrated clips 216. The mechanical features shown in FIG. 3 can help to ensure that the helical section (206) remains substantially vertically aligned above the drain bag (202).

In the illustrative embodiment of FIG. 3, the mechanical features comprise a retainer clip configured to couple to a portion of the drain tubing (201) extending from an end of the helical section (206). This illustrative retainer clip is configured to stiffen the portion of the drain tubing (201) extending from the end, i.e., the bottom, of the helical section (206). The retainer clip in this embodiment comprises two cantilevered arms 302,303 extending distally from the hanger 215 toward each other. An optional tubing support 304 can be included as well.

Turning to FIG. 4, the hanger 215 is shown with the bag connection device 301 coupled to a drain bag 402. Drain tubing 201 is connected to the drain bag 402. The two cantilevered arms 302,303 are coupled to a portion 401 of the drain tubing 201 at the bottom 403 of the helical section 206. In so doing, the two cantilevered arms 302,302 work to orient the drain tubing 201 in a substantially constant alignment above the drain bag 402.

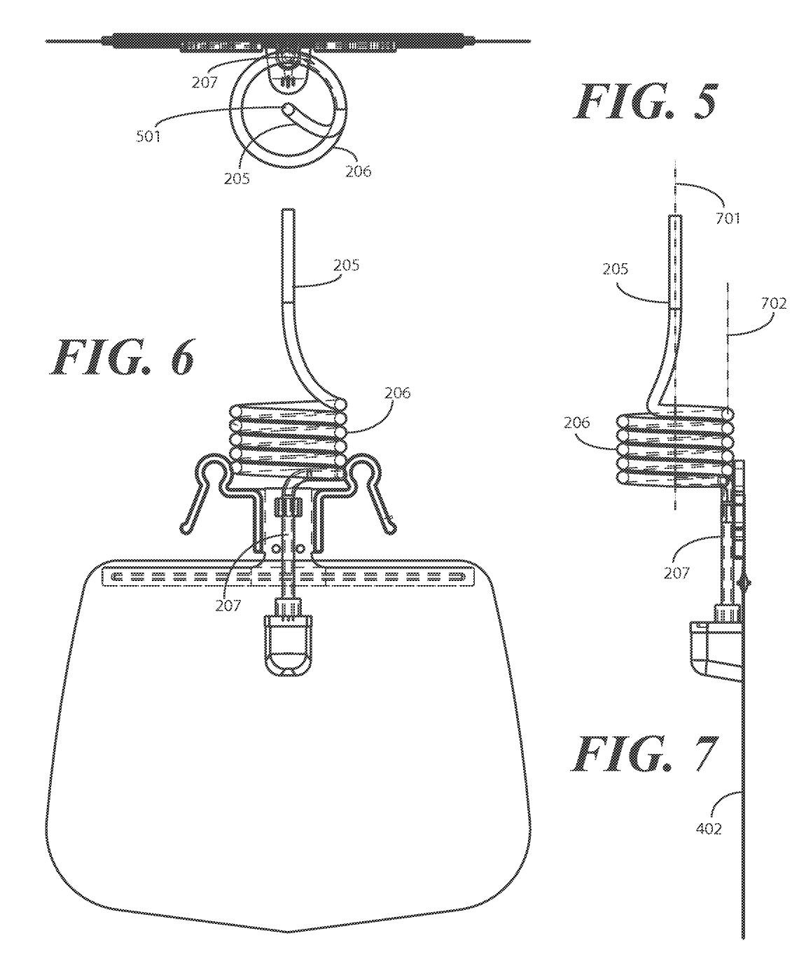

In one embodiment, the helical section 206 has a unique geometric configuration that further helps to maintain an optimal “fluid draining” orientation as well. This geometric configuration can be seen in FIGS. 5, 6, and 7. FIG. 5 is a top plan view, while FIG. 6 is an elevation section view. FIG. 7 is a side section view. FIG. 6 provides a reference for what is shown in FIGS. 5 and 7.

The geometric alignment of the helical section 206 in FIGS. 5, 6, and 7 concerns the entry point of the first elongated section 205 with the helical section 206 and the exit point of the second elongated section 207 from the helical section 206. As shown in these figures, in one embodiment the first elongated section 205 enters the helical section 206 axially, while the second elongated section 207 exits the helical section 206 tangentially.

The axial entry is most easily seen in FIGS. 5 and 7. As shown in FIG. 7, the first elongated section 205 enters the helical section 206 along an axis 701 disposed substantially in the center of the helical section 206. In FIG. 5, this is seen at the section view 501 of the first elongated section 205, which is disposed substantially in the center of the helical section 206. As was described with reference to FIG. 2, in one embodiment the first elongated section 205 extends axially away from the helical section 206 to the catheter connector (203).

The tangential exit is most readily seen in FIG. 7. As shown, the drain tubing of the second elongated section 207 extends from the end 403 of the helical section 206 to the drain bag 402 from a perimeter 702 of the helical section 206. This tangential exit facilitates easy coupling with the retainer clip shown in FIG. 3.

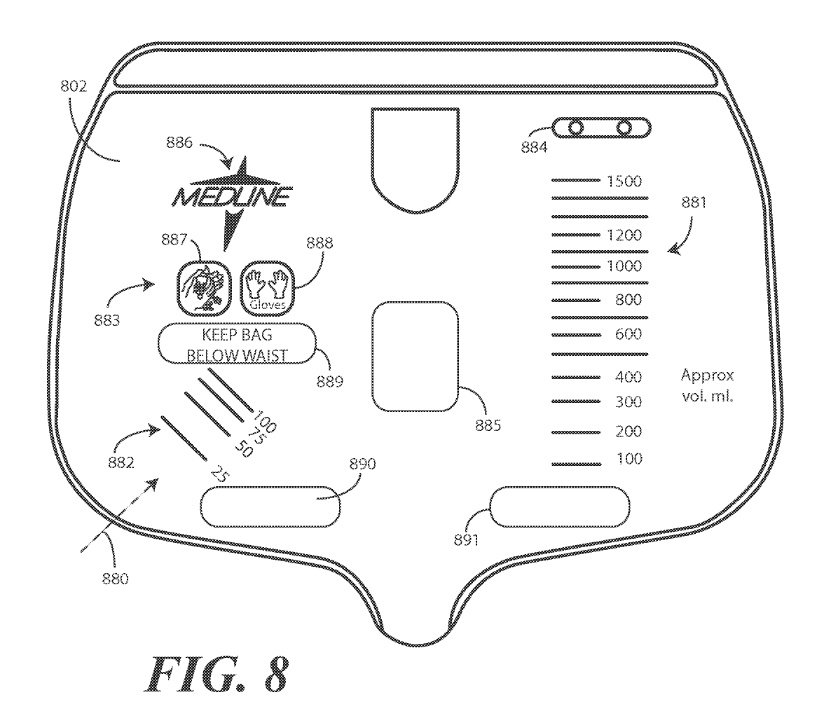

Turning now to FIG. 8, illustrated therein is one illustrative drain bag 802 suitable for use with assemblies and tubing devices in accordance with embodiments of the invention. As noted above, in one embodiment, the drain bag 802 is configured as a low-profile bag having a capacity of about 1500 milliliters or less, which permits the drain bag 802 to have a body only about eight inches in length. In one embodiment, the drain bag 802 is manufactured from a front layer of polyvinyl chloride film that is coupled to a rear layer of polyvinyl chloride film. Standard features can be included, such as an air vent 884, a “dog house” 885, and branding information 886.

The illustrative drain bag 802 of FIG. 8 includes capacity demarcations 881 that indicate the amount of fluid within the drain bag 802 in a vertical orientation. Optional second capacity demarcations 882 indicate the amount of fluid in the drain bag 802 when oriented along a non-vertical axis 880.

Safety information 883 can optionally be included as well. For example, in the illustrative embodiment of FIG. 8, the safety information 883 includes a placard 887 indicating that medical services personnel should wash their hands, a placard 888 indicating that medical services personnel should don gloves, and a placard 889 indicating that the drain bag 802 should remain below the waist of the user.

As noted above, in one embodiment a catheter assembly can be configured with a valve (212) suitable for draining the drain bag 802. As also noted above, in one embodiment the valve (212) is configured as a color-coded slide tap. Where this is the case, valve placards 890,891 can be added to the drain bag 802 to explain and/or illustrate the color-coded system.

Turning now to FIGS. 9 and 10, illustrated therein are examples of valve placards 890,891 along side an illustrative valve 212 configured as a slide tap. Beginning with FIG. 9, the valve placard 890 indicates that when the slide 901 of the valve 212 is pushed out of the body 902 of the valve 212, which is to the left in FIG. 9, the valve is open. This is indicated graphically in the placard 890 with a drawing 903 of the valve 212 in this position and with a plurality of arrows 904,905 indicating the direction to which the slide 901 should be pushed. To further provide a visible indication of the status of the valve 212, the slide 901 can be color-coded. In this case, a portion 906 of the valve 212 is green. When this portion 906 is pushed out of the body 902, a healthcare provider can see “green.” As indicated on the placard 890, this means the valve 212 is open.

Turning now to FIG. 10, illustrated therein is the other placard 891. This placard 891 indicates that when the slide 901 of the valve 212 is pushed into of the body 902 of the valve 212, which is to the right in FIG. 10, the valve is closed. This is indicated graphically in the placard 899 with a drawing 1003 of the valve 212 in this position and with a plurality of arrows 1004,1005 indicating the direction to which the slide 901 should be pushed.

As shown to the left of the placard 891, when the slide 901 is pushed into the body 902, a distal end 1001 becomes exposed. To further provide a visible indication of the status of the valve 212, the distal end 1001 can be color-coded. In this case, the distal end 1001 of the slide 901 is red. When the distal end 1001 is pushed out of the body 902, a healthcare provider can see “red.” As indicated on the placard 891, this means the valve 212 is closed. This color-coding system helps to prevent fluid spillage.

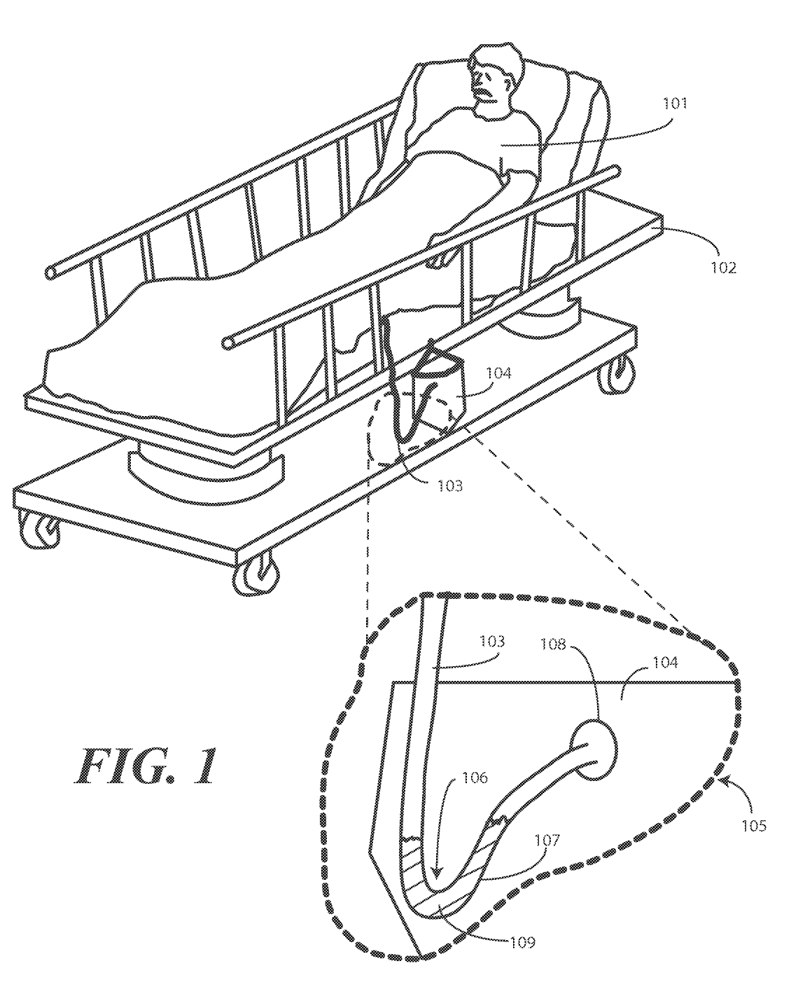

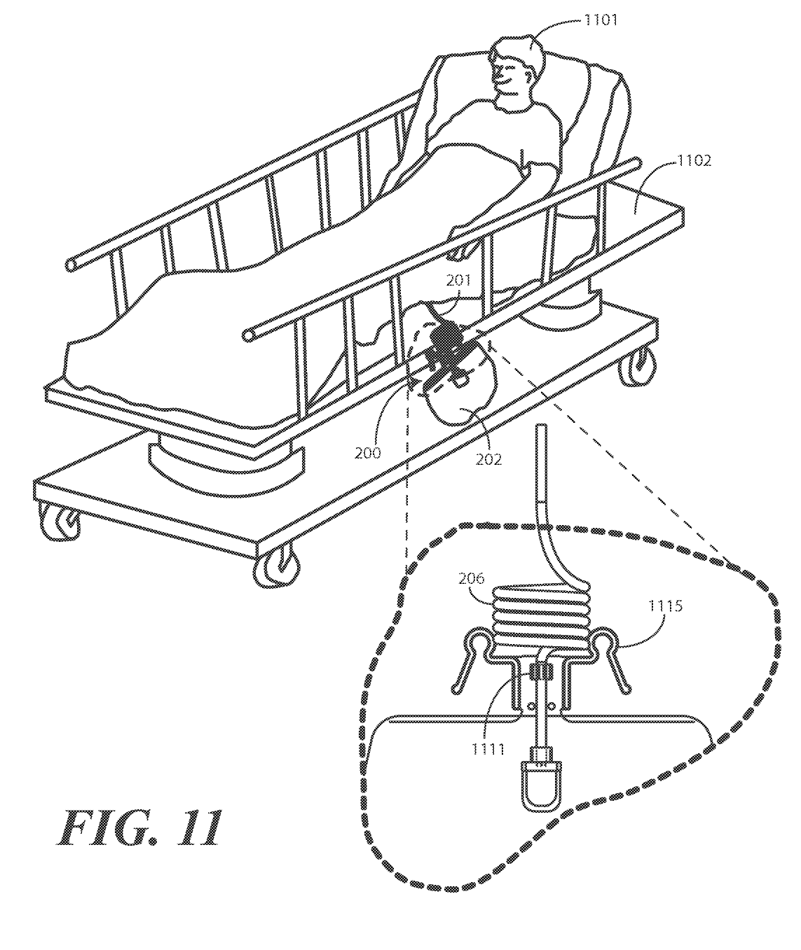

Turning now to FIG. 11, illustrated therein is one example of a catheter assembly 200 configured in accordance with embodiments of the invention when in use. As with FIG. 1, a patient 1101 is confined to a bed 1102 or is otherwise unable to go to the restroom. Also as with FIG. 1, drain tubing 201 is inserted into the patient's urethra. In one embodiment, the drain tubing 201 is held in place by a balloon that is filled with sterile water. The drain tubing 201 then drains urine into a drain bag 202 that is coupled to the patient's bed 1102.

In contrast to FIG. 1, there is no dependent loop in FIG. 11. This is because the drain tubing 201 includes a helical section 206. In this illustrative embodiment, the helical section 206 is stiffened and aligned upright with the assistance of a mechanical connector 1111 on a hanger 1115. The helical section 206 is configured to expand and retract as the patient 1101 moves. However, no dependent loops are formed. Consequently, the risk of pooled urine and/or air-fluid locks is reduced or eliminated due to the mechanical and functional characteristics of the drain tubing 201. In particular, the drain tubing 201 uses gravity to its fullest potential. With the elimination of dependent loops, the need for expensive bacteriostatic or antimicrobial coatings to prevent microbial growth is also eliminated. The inclusion of a helical section 206 not only facilitates flow, but also enables versatility in product placement and use. The adjustable helical section length maintains a direct fluid flow into the drain bag 202 as much of the time as possible.

In the foregoing specification, specific embodiments of the present invention have been described. However, one of ordinary skill in the art appreciates that various modifications and changes can be made without departing from the scope of the present invention as set forth in the claims below. Thus, while preferred embodiments of the invention have been illustrated and described, it is clear that the invention is not so limited. Numerous modifications, changes, variations, substitutions, and equivalents will occur to those skilled in the art without departing from the spirit and scope of the present invention as defined by the following claims. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims.

What is claimed is:

1. A connector for establishing a fluid conduit connection between a catheter and a drain bag, the connector comprising:
a helical section of a flexible tubing material,
a first elongated section of the flexible tubing material extending distally from a first end of the helical section and terminating at a catheter connector; and
a second elongated section of the flexible tubing material extending distally from a second end of the helical section and terminating at a drain bag connector.

2. The connector of claim 1, wherein the helical section is expandable.

3. The connector of claim 2, wherein the helical section is self-retracting.

4. The connector of claim 1, wherein a length of the connector is configured to be alterable by pulling the first elongated section away from the second elongated section, thereby altering an axial length of the helical section.

5. The connector of claim 1, wherein the helical section comprises a plurality of turns.

6. The connector of claim 5, wherein the plurality of turns comprises between three and six turns.

7. The connector of claim 1, wherein:
the first elongated section defines a first fluid-conveying aperture therein;
the helical section defines a helical fluid-conveying aperture therein; and
the first fluid-conveying aperture has a diameter greater than the helical fluid-conveying aperture.

8. The connector of claim 1, wherein the first elongated section is coupled to the helical section by a tubular connector.

9. The connector of claim 1, wherein the flexible tubing material of the helical section has a length of between 60 and 100 millimeters.

10. The connector of claim 9, wherein the first elongated section has an elongated length of between 1000 and 1300 millimeters.

11. The connector of claim 1, wherein the flexible tubing material comprises polyvinylchloride.

12. The connector of claim 1, wherein the catheter connector comprises a sampling port having a locking device configured to lock to a syringe.

13. A urinary catheter assembly, comprising:
drain tubing extending from a catheter connector and comprising a helical section; and
a drain bag configured for attachment to the drain tubing.

14. The urinary catheter assembly of claim 13, further comprising a hanger configured for attachment to the drain bag, the hanger comprising a retainer clip configured to couple to a portion of the drain tubing extending from an end of the helical section.

15. The urinary catheter assembly of claim 14, wherein the retainer clip is configured to stiffen the portion of the drain tubing extending from the end of the helical section.

16. The urinary catheter assembly of claim 15, wherein the retainer clip comprises two cantilevered arms extending distally from the hanger and toward each other.

17. The urinary catheter assembly of claim 13, wherein the drain tubing extends axially from the helical section to the catheter connector.

18. The urinary catheter assembly of claim 17, wherein a portion of the drain tubing extending from an end of the helical section extends to the drain bag from a perimeter of the helical section.

19. The urinary catheter assembly of claim 13, wherein the catheter connector comprises a color-coded valve, wherein a color-coding convention corresponding to the color-coded valve is depicted on the drain bag.

20. The urinary catheter assembly of claim 13, wherein the drain bag has a capacity of about 1500 milliliters or less.

21. The urinary catheter assembly of claim 13, wherein the drain bag comprises a valve.

22. The urinary catheter assembly of claim 21, wherein the valve is configured with a colored indicator indicative of whether the valve is open.

23. The urinary catheter assembly of claim 22, wherein the colored indicator is configured to be red when the valve is closed and green when the valve is open.
